(19) United States
(12) Patent Application Publication
(10) Pub. No.: US 2016/0273047 A1
(43) Pub. Date: Sep. 22, 2016

(54) EPITHELIAL-MESENCHYMAL TRANSITION IN CIRCULATING TUMOR CELLS (CTCS) NEGATIVES FOR CYTOKERATIN (CK) EXPRESSION IN PATIENTS WITH NON-METASTATIC BREAST CANCER

(71) Applicants: SERVICIO ANDALUZ ED SALUD, Sevilla (ES); UNIVERSIDAD DE GRANADA, Granada (ES)

(72) Inventors: José Luis GARCÍA PUCHE, Granada (ES); María José SERRANO FERNÁNDEZ, Sevilla (ES); José Antonio LORENTE ACOSTA, Granada (ES); Juan Antonio MARCHAL CORRALES, Granada (ES); Francisco Gabriel ORTEGA SÁNCHEZ, Sevilla (ES)

(21) Appl. No.: 15/033,385

(22) PCT Filed: Oct. 30, 2014
(86) PCT No.: PCT/EP2014/073405

§ 371 Date: Apr. 29, 2016

(30) Foreign Application Priority Data
Oct. 30, 2013 (EP) 13382436.7

(51) Int. Cl. C12Q 1/68

(52) U.S. Cl. CPC C12Q 1/6886; C12Q 2600/158; C12Q 2600/118; C12Q 2600/106

(57) ABSTRACT

The inventors of the present invention have surprisingly discovered that EGFR expression in nonmetastatic breast cancer patients with CK-negative CTCs could induce EMT process. A simultaneous detection of both EGFR and EMT markers (VIM and Slug) in CTCs might improve prognostic or predictive information in patients with operable breast cancer.

### TECHNICAL FIELD OF THE INVENTION

The present invention relates in general to the field of cancer, and more particularly, to biomarkers for the prognosis of cancer.

### BACKGROUND OF THE INVENTION

Breast cancer is the principal cause of death for women in the world. The development of new diagnostic techniques and the early detection have allowed a decrease of deaths each year; however further improvements are needed. In the last years, detection and characterization of circulating tumor cells (CTCs) has become an active area of translational cancer research. CTCs detection is possible at both early and late stage of cancer development and might allow the estimation of risk relapse and survival, playing a prominent role as prognostic and predictive factor in several types of solid tumors. Especially important is its role in the breast cancer evolution where it has been demonstrated the implication of CTCs in the progression of this disease. Nevertheless, the knowledge of biological properties of CTCs is still limited. It is broadly accepted that during tumorigenesis CTCs acquire features of invasiveness and motility, surviving in hostile environments, such as bloodstream. These phenotypic changes are associated with or at least partially consequence of the epithelial-mesenchymal transition (EMT) phenomenon. The importance of EMT in breast cancer has been established between primary tumor and CTCs expressing EMT markers. This association was higher in metastatic breast cancer patients than in early breast cancer patients, suggesting that EMT phenotype is directly related with metastatic potential of CTCs. The process of EMT involves the formation of metastatic cancer cells that gain expression of mesenchymal markers such as vimentin (VIM) or Slug and loss of epithelial markers including EpCAM or CK, acquiring a mesenchymal or semimesenchymal phenotype. However, the presence of this EMT phenotype has been associated not only with the metastatic potential of CTCs but also with the capacity of these CTCs to present drug resistance.

Vimentin is a type III intermediate filament protein expressed in cells of mesenchymal origin. Nevertheless, its expression has been described in epithelial cells from pathologic process and is characteristically up-regulated in cells undergoing EMT. Vimentin expression in epithelial tumor cells undergoing EMT process is related to a reduced expression of E-cadherin, N-cadherin upregulation and the enhancement of tumor cells migration and invasiveness. Moreover, high levels of VIM expression in cancer patients are correlated with poor prognosis, and the simultaneous expression of VIM and CK in breast tumor cells might be associated with poorer survival in breast cancer patients.

Expression of some transcription factor such as Slug has been associated with a poor prognosis in a variety of human cancers. The encoded protein acts as a transcriptional repressor of E-cadherin that binds to E-box motifs in breast carcinoma. Moreover, Slug is involved in EMT, has antiapoptotic activity and has been associated with resistance to chemotherapy in ovarian carcinomas. Various mechanisms underlie the regulation of this transcriptional factor and interplay in its effect on tumor progression and invasiveness. However, the molecular events implicated in the regulation of this EMT process remain unexplored.

EGFR is a member of the EGFR/ErbB/HER family of Type I transmembrane tyrosine kinase receptors, which include ErbB2/HER-2/neu, ErbB3/HER-3 and ErbB4/HER-4. The ErbB receptors play an essential role in organ development and growth by regulating both the differentiation and morphology of cells and tissues. EGFR signaling is important in normal epithelial development and in tumor cell proliferation, motility, survival and metastasis and it is known to be overexpressed in breast cancer, especially in triple negative breast cancer. Several studies have demonstrated that tyrosine kinase inhibitors (TKIs) targeting EGFR are effective in a variety of cancers. Unfortunately, patients eventually develop resistance to these agents and EMT markers expression might be involved in acquiring resistance to EGFR-TKI. Recently, it has been found that EGFR can interact with the cell adhesion molecule E-cadherin, which is related to EMT process. In addition, EGFR inhibition suppressed EMT in human pancreatic cancer cell line PANC-1 and consequently decreased cell migration and invasion ability.

On the other hand, CD133 is known to be a marker of primitive hematopoietic stem and progenitor cells. In breast cancer, cells displaying a stem cell-like gene expression profile overexpress cancer stem cell markers, such as CD133, CD44 and ALDH1. Moreover, CD133 plays an important role in migration and invasion by facilitating EMT.

To date no one has studied the relationship of EGFR expression with EMT markers such as VIM and Slug in CTCs of cancer patients, in particular breast cancer patients, lung cancer patients, colon cancer patients and prostate cancer patients. Since EGFR may promote proliferation and progression in cancer and VIM and Slug are potential regulators of cell adhesion and migration, the inventors aimed to determine the correlation between the expression of these biomarkers and the up EGFR expression in CTCs negatives for CK. Also, the inventors studied the ability of these CK-CTCs with EMT features to predict prognosis in cancer patients.

### BRIEF DESCRIPTION OF THE INVENTION

A first aspect of the invention refers to a method for prognosticating cancer in a human subject with cancer, preferably with a solid tumour of epithelial origin, more preferably with a non-metastatic solid tumour of epithelial origin, comprising the steps of:
a. obtaining one or more biological samples from the subject suffering from cancer comprising circulating tumour cells;
b. measuring the overall expression pattern or level of the following biomarkers in the circulating tumour cells obtained from the one or more biological samples of the subject: CK (cytokeratin) and EGFR (epidermal growth factor receptor); and
c. comparing the overall expression pattern of the above mentioned biomarkers in the circulating tumour cells from the biological sample of the subject suffering cancer with the overall expression pattern of the biomarkers from a control biological sample, wherein expression of EGFR in CK negative (underexpressed) circulating tumour cells is indicative of Progression Free Survival (PFS) and/or Overall Survival (OS).

Control biological sample is understood in the present invention as any blood cell from a healthy donor.

Expression in the context of the present invention is understood as absence or presence of the biomarker.

A CK negative circulating tumour cell in the context of the present invention is understood as absence of the CK biomarker in said cell or an expression in said cell lower than ⅓ of the normal expression in blood cells from healthy donors.

A second aspect of the invention refers to a method for prognosticating cancer in a human subject with cancer, preferably with a solid tumour of epithelial origin, more preferably with a non-metastatic solid tumour of epithelial origin comprising the steps of:
a. obtaining one or more biological samples from the subject suffering from cancer comprising circulating tumour cells;
b. measuring the overall expression pattern or level of the following biomarkers in the circulating tumour cells obtained from the one or more biological samples of the subject: CK (cytokeratin) and EMT antigens VIM and Slug; and
c. comparing the overall expression pattern of the above mentioned biomarkers in the circulating tumour cells from the biological sample of the subject suffering from cancer with the overall expression pattern of the biomarkers from the circulating tumour cells in a control biological sample, wherein overexpression of EMT antigens VIM and Slug or increase expression in CK negative (underexpressed) circulating tumour cells is indicative of Progression Free Survival (PFS) and/or Overall Survival (OS).

A third aspect of the invention refers to a method for prognosticating cancer in a human subject with cancer, preferably with a solid tumour of epithelial origin, more preferably with a non-metastatic solid tumour of epithelial origin comprising the steps of:
a. obtaining one or more biological samples from the subject suffering from cancer comprising circulating tumour cells;
b. measuring the overall expression pattern or level of the following biomarkers in the circulating tumour cells obtained from the one or more biological samples of the subject: CK (cytokeratin), EGFR (epidermal growth factor receptor) and EMT antigens VIM and Slug; and
c. comparing the overall expression pattern of the above mentioned biomarkers in the circulating tumour cells from the biological sample of the subject suffering from cancer with the overall expression pattern of the biomarkers from the circulating tumour cells in a control biological sample, wherein overexpression or increase expression of EMT antigens VIM and Slug and of EGFR in CK negative (underexpressed) circulating tumour cells is indicative of Progression Free Survival (PFS) and/or Overall Survival (OS).

In a preferred embodiment of any of the precedent aspects, the one or more biological samples are selected from the group consisting of a plasma sample, a serum sample, a blood sample, a tissue sample, and a fecal sample.

In a preferred embodiment of any of the precedent aspects, the expression level of the biomarkers is measured by microarray expression profiling, PCR, reverse transcriptase PCR, reverse transcriptase real-time PCR, quantitative real-time PCR, end-point PCR, multiplex end-point PCR, cold PCR, ice-cold PCR, mass spectrometry, in situ hybridization (ISH), multiplex in situ hybridization, or nucleic acid sequencing.

In a preferred embodiment of any of the precedent aspects, the cancer is a solid tumour of epithelial origin, in particular the cancer is a non-metastatic solid tumour of epithelial origin, more particularly the cancer is selected from the list consisting of colon cancer, lung cancer, breast cancer and prostate cancer.

In a preferred embodiment of any of the precedent aspects and of any of its preferred embodiments, the method is used for treating a patient suffering from cancer, selecting an anti-neoplastic agent therapy for a patient suffering from cancer, stratifying a patient to a subgroup of cancer or for a cancer therapy clinical trial, determining resistance or responsiveness to a cancer therapeutic regimen, developing a kit for diagnosis of cancer or any combinations thereof.

In a preferred embodiment of any of the precedent aspects and of any of its preferred embodiments, the method further comprises the step of using the overall expression pattern or level of the biomarkers for prognosis, treatment guidance, or monitoring response to treatment of the cancer.

A fourth aspect of the invention refers to a kit for a prognosis of cancer or for predicting the response of a human subject suffering from cancer, preferably with solid tumour of epithelial origin, more preferably with a non-metastatic solid tumour of epithelial origin, more preferably with a cancer selected from the list consisting of colon cancer, lung cancer, breast cancer (non-metastatic breast cancer) and prostate cancer, to therapy with an EGFR inhibitor comprising: biomarker detecting reagents for determining a differential expression level of EMT antigens VIM and Slug, EGFR and CK in circulating tumour cells obtained from biological samples.

The kit of the fourth aspect of the invention, further comprising instructions for use in diagnosing risk for cancer, wherein the instructions comprise step-by-step directions to compare the expression level of the biomarkers, when measuring the expression of a sample obtained from a subject suspected of having ca neoplasia with the expression level of a control sample.

The kit of the fourth aspect of the invention or of any of its preferred embodiments, further comprising tools, vessels and reagents necessary to obtain samples from a subject selected from the group consisting of one or more biological fluids, a plasma sample, a serum sample, a blood sample, a tissue sample, or a fecal sample.

A fifth aspect of the invention refers to a method of predicting the response of a human subject to therapy with an EGFR inhibitor, wherein the subject is suffering from cancer, wherein the cancer is preferably a solid tumour of epithelial origin, more preferably the cancer is a non-metastatic solid tumour of epithelial origin, more particularly the cancer is selected from the list consisting of colon cancer, lung cancer, breast cancer and prostate cancer, comprising the steps of:
a. obtaining one or more biological samples from the subject suffering from cancer comprising circulating tumour cells;
b. measuring the overall expression pattern or level of the following biomarkers in the circulating tumour cells obtained from the one or more biological samples of the subject: CK (cytokeratin) and EGFR (epidermal growth factor receptor); and
c. comparing the overall expression pattern of the above mentioned biomarkers in the circulating tumour cells from the biological sample of the subject suffering from cancer with the overall expression pattern of the biomarkers from a control biological non-cancerous sample, wherein overexpression or increase expression of EGFR in CK negative (underexpressed) circulating tumour cells is indicative of no response and/or partial response of the subject.

A sixth aspect of the invention refers to a method of predicting the response of a human subject to therapy with an EGFR inhibitor, wherein the subject is suffering from cancer, wherein the cancer is preferably a solid tumour of epithelial origin, more preferably the cancer is a non-metastatic solid tumour of epithelial origin, more particularly the cancer is selected from the list consisting of colon cancer, lung cancer, breast cancer and prostate cancer, comprising the steps of:
a. obtaining one or more biological samples from the subject suffering from cancer comprising circulating tumour cells;
b. measuring the overall expression pattern or level of the following biomarkers in the circulating tumour cells obtained from the one or more biological samples of the subject: CK (cytokeratin) and EMT antigens VIM and Slug; and
c. comparing the overall expression pattern of the above mentioned biomarkers in the circulating tumour cells from the biological sample of the subject suffering from cancer with the overall expression pattern of the biomarkers from the circulating tumour cells in a control biological sample, wherein overexpression of EMT antigens VIM and Slug or increase expression in CK negative (underexpressed) circulating tumour cells is indicative of no response and/or partial response of the subject.

A seventh aspect of the invention refers to a method of predicting the response of a human subject to therapy with an EGFR inhibitor, wherein the subject is suffering from cancer, wherein the cancer is preferably a solid tumour of epithelial origin, more preferably the cancer is a non-metastatic solid tumour of epithelial origin, more particularly the cancer is selected from the list consisting of colon cancer, lung cancer, breast cancer and prostate cancer, comprising the steps of:
a. obtaining one or more biological samples from the subject suffering from cancer comprising circulating tumour cells;
b. measuring the overall expression pattern or level of the following biomarkers in the circulating tumour cells obtained from the one or more biological samples of the subject: CK (cytokeratin), EGFR (epidermal growth factor receptor) and EMT antigens VIM and Slug; and
c. comparing the overall expression pattern of the above mentioned biomarkers in the circulating tumour cells from the biological sample of the subject suffering from cancer with the overall expression pattern of the biomarkers from the circulating tumour cells in a control biological non-cancerous sample, wherein overexpression or increase expression of EMT antigens VIM and Slug and of EGFR in CK negative (underexpressed) circulating tumour cells is indicative of no response and/or partial response of the subject.

In a preferred embodiment of any of the fifth, six, or seventh aspects of the invention, the EGFR inhibitor is selected from the group consisting of Gefitinib, Erlotinib, Cetuximab, lapatinib, pannitumumab and trastuzumab.

An eighth aspect of the invention refers to a method for allocating a human subject suffering from cancer in one of two groups, wherein group 1 comprises subjects identifiable by the method according to any of the fifth, six, or seventh aspects of the invention, as predicted to show any one or more of:
(i) No response; or
(ii) partial response;
and wherein group 2 represents the remaining subjects.

A ninth aspect of the invention refers to a pharmaceutical composition comprising an anti-EGFR agent, for treating a human subject of group 2 as identifiable by the method of the precedent aspect of the invention. Preferably the anti-EGFR agent is selected from the group consisting of Gefitinib, Erlotinib, Cetuximab, lapatinib, pannitumumab and trastuzumab.

### BRIEF DESCRIPTION OF THE FIGURES

FIG. 1. Schema of the methodology used to assess the characterization by IF of CK negative samples. Patients donated three 10 ml samples of peripheral blood at the time of initial diagnosis. Equal samples were obtained from healthy volunteers. In each tube CTCs negatives to CK expression were further evaluated for VIM/Slug expression and for EGFR (A), TOP2A/HER2 (B) and CD133 (C) expression, respectively.

FIG. 2. Epifluorescence microscopy images of stained cells according their expression of VIM and Slug. HUVEC cells used as positive control to VIM (FITC, green) and Slug (Alexa633, Red). Nuclei were stained in blue (DAPI) (A). Representative images of the different cellular distribution of Slug, cytoplasmic (B) or nuclear (C), in CTCs CK-negative/VIM⁺/Slug⁺ from breast cancer patients.

FIG. 3: Effects of TGFβ1 and EGF on EMT, apoptosis and CK expression in MCF-7 cells. MCF-7 cells were treated with TGFβ1 (0.5 and 10 ng/ml), EGF (20 ng/ml), or a combination of TGFβ1 and EGF (5 or 10 ng/ml and 20 ng/ml, respectively) for 72 hours. (A) Western blots analysis using anti-EGFR, anti-phospho EGFR, anti-Slug, anti-VIM, anti-Bcl-2, anti-caspase 9, anti-multi-CK, anti-TGFβ R2 anti-β-actin antibodies. β-actin was used as loading control. (B) Densitometric analysis related to control. (C) Luminescence detection of Caspase-3, -7 and -8 activities. Enzymatic activity was expressed in relative light units (RLU). Data represent the mean±SD from three independent experiments (*p<0.05, ***p<0.01 vs. control). CTC CK-negative/EGFR⁺/VIM⁺/Slug⁺ sample (C). Note: nuclei of the cells were stained with DAPI after the visualization of VIM, EGFR and Slug expression.

FIG. 4. Expression of EGFR, VIM, Slug and CK in MCF-7 cells after TGFβ1 and/or EGF induction. Confocal microscopy examination of MCF-7 cells treated with TGFβ1 (0 and 10 ng/ml), EGF (20 ng/ml), or a combination of TGFβ1 and EGF (10 ng/ml and 20 ng/ml, respectively) for 72 h. Representative immunofluorescence and light microscopic images of EGFR, VIM, Slug and CK staining. (A) Non-treated control cells showed marked staining for CK and a low EGFR expression. (B) TGFβ1-treated cells displayed the expression of VIM and Slug and a decrease in CK staining. (C) TGFβ1/EGF combined treatment induced a high expression of EGFR, VIM and Slug and the total disappearance of CK. Nuclei are stained with DAPI (blue). Original magnification: All images at 60×.

FIG. 5. Wound healing assays in MCF-7 cells after induction with TGFβ1 and/or EGF. (A) Migration of the cells to the wound was visualized at 0, 24 and 48 h with an inverted phase-contrast microscope (100× magnification). (B) Quantification of cell migration was done by counting the free pixels inside and outside of the detection zone (Data were analyzed as percentages of the control cells in three independent experiments. *p<0.05 and **p<0.01 were considered significant).

FIG. 6. VIM and Slug expression in EGFR positive cells. SKBR cells used as positive control for EGFR and negative for VIM and Slug. Representative images of the different phenotypes found in CK-negative/EGFR+CTCs from BC patients. CTC CK-negative/EGFR+/VIM+/Slug− sample. CTC CK-negative/EGFR+/VIM+/Slug+ sample. Note: nuclei of the cells were stained with DAPI after the visualization of VIM, EGFR and Slug expression. Original magnification 40×

FIG. 7. Mechanisms by which TGFβ1 and EGFR activation may act to enhance the EMT response in CTCs negatives for CK of breast cancer patients.

FIG. 8. A. Image galleries after isolation and cytomorphological analysis and CTC− EMT positives cells (EGFR (1)), Vim (2), Slug (3) and nucleous (4) detection in patients with solid tumor. A. CTC, from a colon cancer patient B. CTC from lung cancer patient .C. CTC from a prostate cancer patient.

### DETAILED DESCRIPTION OF THE INVENTION

In the context of the present invention, the following abbreviations have been used: BC: breast cancer; CTCs: circulating tumor cells; ER: estrogen receptor; PR: progesterone receptor; EGFR: epidermal growth factor receptor; IF: immunofluorescence; AT: adjuvant therapy; NAT: neoadjuvant therapy; HR: hormone receptors; IHC: immunohistochemical; CK: cytokeratin; EMT: epithelial-mesenchymal transition; EGFR: growth factor receptor, VIM: vimentin.

The systemic nature of breast cancer is defined by the dissemination of early tumor cells, even with relatively small tumors. In this scenario, the metastatic process involves the dissemination of CTCs through the blood and lymphatic system prior to the colonization of distant organs. Several studies have observed the presence of CTCs in peripheral blood as the pre-stadium of clinically manifest distant metastases.

The metastatic process comprises phenotypic alterations that are mediated by genetic changes. Among these phenotypic variations the EMT process modulates cell survival, migration and resistance to anoikis and apoptosis. For EMT process, epithelial cells lose their characteristics and acquire a mesenchymal phenotype, which include both CK or E-cadherin downregulation and VIM or N-cadherin upregulation, respectively.

Nowadays, the majority of techniques used to isolate and detect CTCs are based in the expression of epithelial markers. Consequently, if EMT process is essential to migration and survival of tumor cells before the colonization of target organs, then, an important aggressive subpopulation of tumor cells may not be detected. Recently, it has been showed that patients with poor prognostic factors had non-detectable CTCs using routinely techniques.

In the present invention, the inventors evaluated the expression of EMT markers in a cohort of primary breast cancer patients, which were negative for CK in the basal analysis. Moreover, the inventors further explored if EGFR expression in these patients was correlated with the acquisition of the EMT phenotype. To test this hypothesis that an important cell tumor fraction is not detected and, therefore, can give rise to false-negative samples, the inventors analyzed VIM and Slug in CK-negative CTCs in breast cancer patients. Around 27 and 24 percent of negative samples for CK expression were positive for VIM and Slug, respectively. In addition, the experiments conducted by the present inventors showed that in most of the CK-negative CTCs, Slug and VIM were co-expressed. Accordingly, in an in vivo model with transplantable human breast tumor cells uniquely capable of spontaneous EMT events, it has been showed that in primary xenografts tissues Slug was overexpressed in VIM⁺ areas. These results indicate the need to optimize both isolation and detection methods including the surface EMT markers. New methods based in isolating by size may be a powerful tool to detect different CTCs subpopulations.

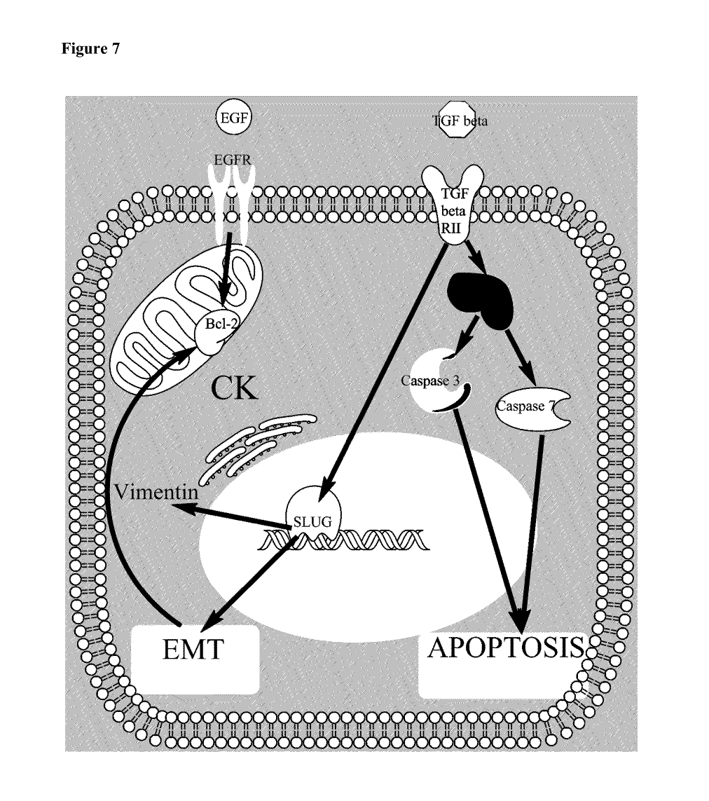

Interestingly, the inventors found a direct relation of tumor size with VIM⁺ and Slug⁺/CK-negative CTCs. Recently, it has been demonstrated that EMT changes are responsible of increased tumor growth and metastasis in prostate and breast cancer xenografts experiments. Moreover, in prostate carcinoma the increase of E-cadherin epithelial marker was inversely correlated with size of the metastasis and this expression was increased compared to the primary lesion. The present results suggest the important role of VIM/Slug EMT markers in the tumor growth of primary breast cancer. Moreover, the present results suggest the important role in cancer in general as illustrated in FIG. 7 wherein similar results to the ones obtained from primary breast cancer were obtained with metastatic colon cancer, lung cancer and prostate cancer.

In relation with Slug cell distribution, the inventors found in most of the samples a cytoplasmic staining. Although, it has been broadly established that Slug shows a nuclear staining pattern, a recent study demonstrated that cytoplasmic Slug induces invasive finger-like protrusions termed invadopodia in pancreatic tumors cells by intracellular F-actin polymerization. This process of modulation in the cytoskeletal structure is directly related with a higher invasive and metastatic capacity. Further studies about this finding are warranted to elucidate the mechanisms of nuclear to cytoplasmic translocation of Slug and its role in the metastatic breast cancer process.

Several authors have correlated EGFR expression with poor prognosis in breast cancer patients and it has been demonstrated that EGFR pathway controls several important biological processes, including cellular proliferation, angiogenesis and inhibition of apoptosis. The EMT program is associated with cellular pathways that confer new characteristics to the cells, such as apoptosis resistance, migration capacity and chemo and radio-resistance. In this sense, the inventors found a statistically significant correlation between EGFR⁺ CTCs and CK⁻/VIM⁺/Slug⁺ CTCs, where around 40% of patients had CTCs with both phenotypes. In the same context, a recent study showed that HER2 overexpressing breast cancer cells is accompanied by partial EMT-like transition through the activation of Wnt/β-catenin signaling pathway leading to transactivation of EGFR and promoting EMT-like transition. Also, it has been reported that high EGFR expression induced EMT, with subsequent Twist, Snail and Slug induction. However, not clinical studies have been performed about relationship between EGFR status, EMT phenotype and CTCs in non-metastatic breast cancer, since it is extremely complicate to characterized CTCs during EMT process. In this sense, the results shown herein suggest that CK-negative CTCs with high EGFR expression induced EMT, and this phenotypic transition could involve EGFR-mediated activation of VIM and subsequent VIM-activated Slug gene expression. (FIG. 7).

To strengthen this hypothesis, the inventors determined the role of EGFR in mediating EMT and CK expression on the MCF7 tumor cell line that posses epithelial characteristics. MCF7 was stimulated with TGFβ₁, a potent initiator of mesenchymal transformation, or TGFβ₁/EGF to induce EMT phenotype. The inventors found that the activation of EGFR up-regulated VIM and Slug mesenchymal markers and down-regulated pan-CK epithelial markers. These results suggest that activation of EGFR signaling by its ligand and the presence of TGFβ₁ induces EMT and subsequently inhibits CK expression (FIG. 7).

Recently, it has been demonstrated that CTCs from patients with metastatic breast cancer had predominantly mesenchymal phenotypes and that EGF can induce EMT-like effects including upregulation of Twist through the EGFR pathway, which is in agreement with the present experimental data. Interestingly, the inventors also detected that cells treated with TGFβ₁ alone showed a dose-dependent increased caspase 3/7 and 8 activities and, in contrast, TGFβ₁/EGF combination overexpressed Bcl-2 and significantly decreased these caspases in MCF-7/EMT⁺/CK-negative cells. TGFβ₁-induced apoptosis has been considered to be largely dependent on caspase activation and, in epithelial cells, TGFβ₁ is able to induce both cell apoptosis and EMT in the same cell type in a cell cycle-related manner in which apoptosis took place at G2/M phase and EMT in G1/S phase. Moreover, the EGFR activation in TGFβ₁ treated cells was sufficient to increase EMT phenotypes, to inhibit apoptotic events and to induce the loss of CK expression (FIG. 7). Loss of epithelial cell marker expression occurs concomitantly with, and as a driver of, wound-healing response and the lost of their defined cell cell-basement membrane contacts and their structural/functional polarity. Signaling through the EGF receptor is known to influence the apoptotic resistance and invasive potential of certain cancers, and this may be associated with the adoption of a transdifferentiated phenotype. In human renal cells, it has been demonstrated that EGF promotes EMT by offsetting the pro-apoptotic effects of TGFβ₁ without preventing its EMT-inducing effect, thereby facilitating the improved survival of cells undergoing EMT, which is in concordance with the results obtain herein in the EGFR activated human breast cancer cells. Moreover, in squamous carcinoma both overexpression of Bcl-2 or EGFR activation induced EMT promoting cell migration and invasion via ERK1/2 and PI3K-regulated MMP-9/E-cadherin signaling. These experimental data could explain the relapse of the disease in the CK negative/EGFR⁺ CTCs of non-metastatic breast cancer patients. However, further studies with a higher number of patients are needed to confirm this hypothesis.

On the other hand, in a recent study, they found a correlation between the development of resistance to gefitinib and Slug expression in non-small cell lung cancers patients with EGFR mutation. Additionally, Slug promoted the invasiveness of tumor cells thought suppression of E-cadherin. In fact, though we did not correlate VIM and Slug expression after receiving systemic treatment with PFS, we found a poor clinical outcome when EGFR expression in CK-negative CTCs was present after systemic treatment. These results suggest that EGFR expression in CTCs is necessary for progression of the disease and that EMT markers might lead the survival of CTCs into peripheral blood.

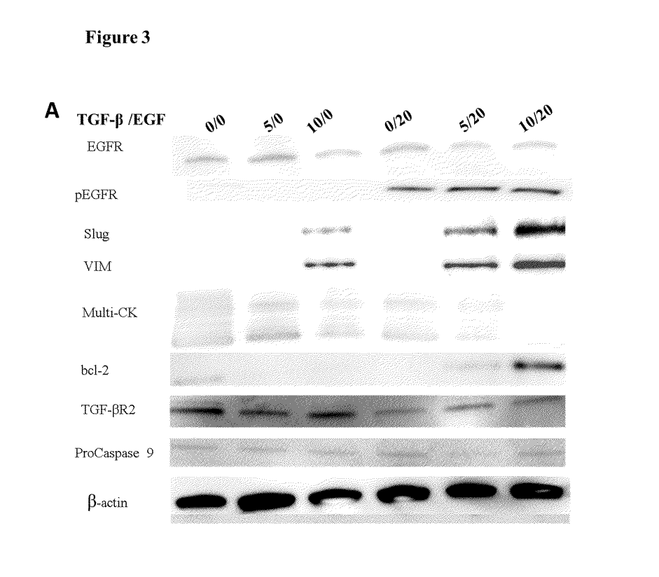

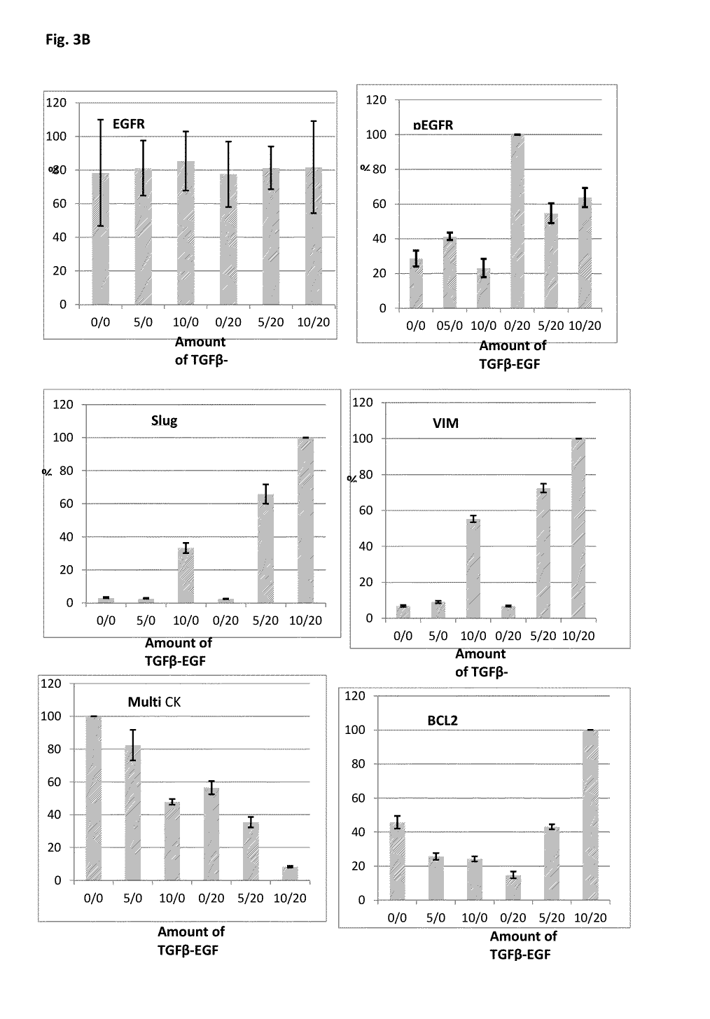

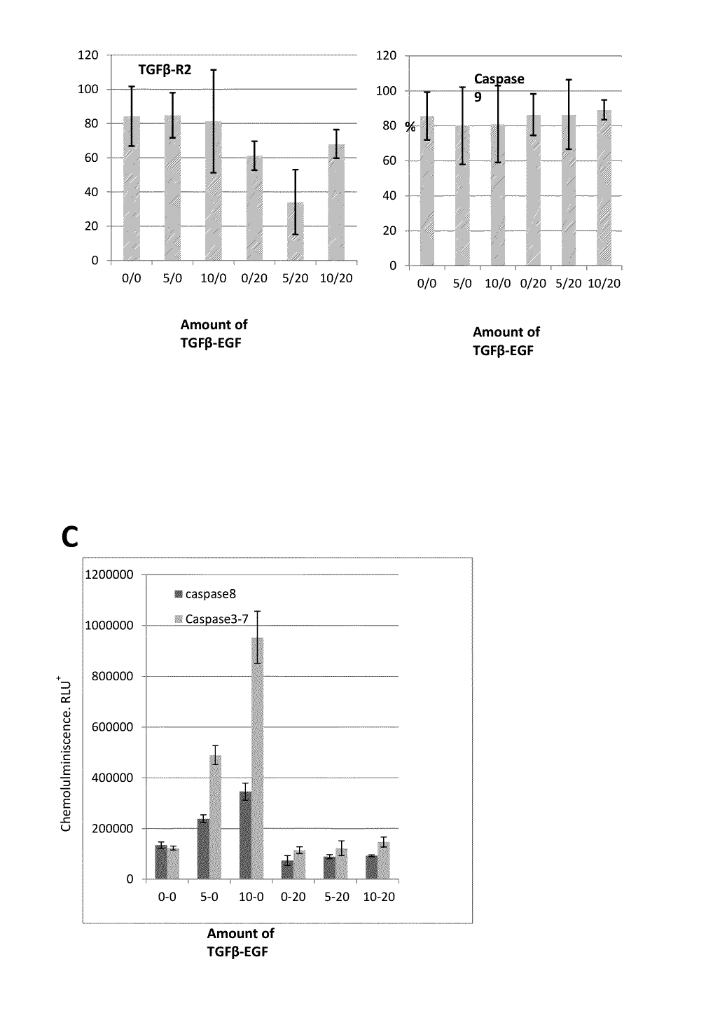

However, the dissemination process is still more complex because the inventors found CTCs in our samples with three different phenotypes: CK⁺/EGFR⁺/Vim⁻/Slug⁻; CK⁻/EGFR⁺/Vim⁻/Slug⁻; CK⁻/EGFR⁺/Vim⁻/Slug⁺ (FIG. 3). Moreover, the inventors found that patients CK⁻/EMT+ at baselines were CK⁺ after treatment. In fact, it has been showed that after targeted therapy, CTCs from responding breast cancer patients were fewer in numbers and had a more epithelial phenotype and conversely, CTCs from refractory patients were more numerous and retained or acquired a mesenchymal phenotype. In these patients with metastatic breast cancer were detected a significant number of CTCs exhibiting a partial or a full-blown EMT phenotype, supportive of an EMT-driven mechanism. Interestingly, a large fraction of the CTCs were either double epithelial/mesenchymal− or mesenchymal-positive, particularly among the HER2⁺ and triple negative subtype. This could be explained by the dynamic plasticity (induction and reversion of EMT) in CTCs, because an epithelial phenotype is favorable in the latter stages of the metastatic cascade and in the metastasis growth. It has been suggested that EMT occurs in the primary tumors, providing the cells with an enhanced ability to intravasate and generate CTCs. MET phenomena would occur to favor the metastatic growth in secondary organs, emphasizing the transient and reversible nature of EMT processes. All our results have been obtained in patients with negative samples for CK expression.

We show that is extremely necessary the use of EMT markers to detect potential aggressive tumor cells with diverse phenotypes. These changes in the cell phenotype could be the key of new therapeutic targets or more effective therapeutic regimens. Furthermore, these results could be indicating that different signaling pathways are activated in CTCs to promote EMT, leading not only the possibility of progression but also survival in a hostile microenvironment. The present findings indicate that a simultaneous detection of EGFR, EMT antigens (VIM and Slug) and CK in CTCs by enrichment methods, such as but not limited to immunomagnetic Separation Techniques, size-based cell enrichment by filtration and cell Density-based Enrichment, contribute to better detection of CTC subpopulation and improve prognostic or predictive information during systemic therapy in patients with operable breast cancer.

As illustrated in example 7, the present findings are not restricted to breast cancer but can be perfectly extrapolated to any type of cancer disease.

The following examples merely illustrate but do not limit the present invention.

### EXAMPLES

### Example 1

### Material and Methods

1.1. Patients

Breast cancer patients with stage I to IIIC were identified from the Breast Cancer Unit of the University Hospital of Jaen and Hospital del Mar of Barcelona from March 2009 to September 2010. The inclusion criteria were histological diagnostic of breast cancer with availability of tissue for biomarker studies. The local ethics committee approved this study and eligible patients signed an approved informed consent. Surgical procedure and systemic therapy were given at the discretion of the treating physician with or without targeted therapy, namely trastuzumab for HER2⁺ breast cancer patients. Medical charts of these patients were reviewed and clinical details were included in a database.

The inventors used a combination of IHC markers for classification of breast cancer patients based on the pattern of expression of hormonal receptors (HR) and HER2 that identify three major distinct molecular breast cancer subtypes luminal tumors, which are HR positive and HER2⁻, HER2 amplified tumors and those tumors with lack of expression of the three receptors.

Tumor specimens from archival tumor biopsies of these patients were obtained and analyzed for different markers. ER and Progesterone Receptor (PR) were assessed by immunohistochemistry (IHC) following ASCO/College of American Pathologists guidelines. HER2 status was determined by IHC using Herceptest (Dako) or FISH when indicated (Pathvysion HER2 DNA Probe Kit from Abbott Molecular; Abbott Park, Ill., USA) following current recommendations [33]. Ki-67 was assessed using mouse monoclonal antibody MIB-1 (1:200 dilutions; Dako, Glostrup, Denmark) and percentage of positively stained nuclei was calculated. Samples with any degree of p53 nuclear staining (clone DO-7, Novocastra Lab, Newcastle, UK) were considered positive.

1.2. CTCs Enrichment

Patients donated three samples of 10 ml of peripheral blood at the time of first diagnostic. Control blood samples were drawn from 16 healthy volunteers with no history of malignant disease. 30 ml of blood were collected from each donor into three different Cell Save Preservatives blood collection Tubes (Vendex, LLC, Johnson & Johnson Company). Blood samples were maintained at room temperature in parallel within a maximum of 72 hours after collection and processed according to the protocol established for our group. Briefly, the samples were processed with a double density gradient methodology (Histopaque 1119 and Ficoll 1077) (Sigma Aldrich). For CTCs enrichment, we used the Carcinoma Cell Enrichment and Detection kit, MACS technology (Miltenyi Biotec). It was performed by selective immunomagnetic cell separation, using magnetic beads labeled with a multi-CK-specific antibody (CK3-11D5) that recognizes CK 7, 8, 18 and 19. Control experiments for the sensitivity and the specificity of this antibody have been reported previously. The cytomorphological criteria proposed by Meng et al. (for example, high nuclear/cytoplasm ratio, larger cells than white blood cells) were used to characterize a CK-positive cell as a CTC. Patients were considered CTCs-positive if were detected ≧1 CTC per 10 ml blood. Samples negatives for CK after selective immunomagnetic cell separation were considered as CK-negative patients.

1.3. Cell Cultures and Immunocytochemistry Assay Feasibility

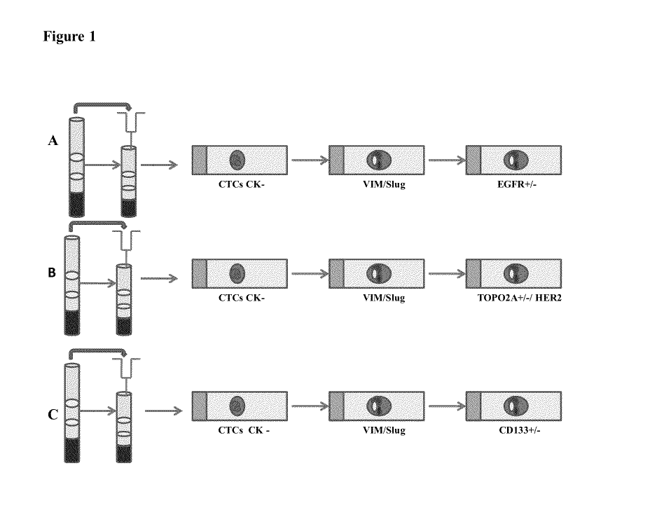

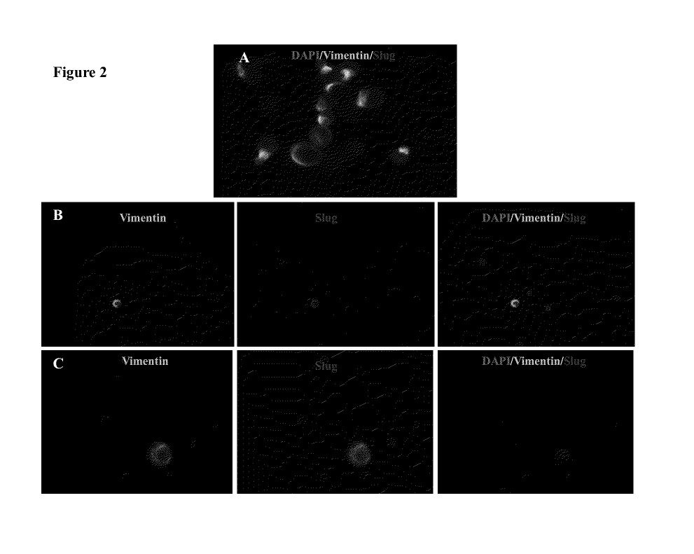

To demonstrate the feasibility for isolation and detection of CTCs we blend peripheral blood with several human breast cancer cell lines as previously reported. Breast cancer cell lines were obtained from the European Collection of Cell Cultures (ECACC, Salisbury, UK). In the recovery experiments, we analyzed control samples with low numbers (10, 5, 1 cells) from MCF-7 (ECACC), SKBR3 (ECACC) and T47D (ECACC) human breast cancer cell lines. Cells were spiked in 10 ml of venous blood from healthy volunteers and control experiments were performed at least in triplicate. Cytospins were prepared afterward by density gradient centrifugation and by immunomagnetic selection in the same way that the patient samples. Recovery rates of tumor cells spiked into normal blood at the low level control numbers were in the range of 40-60%. As negative controls, 16 blood samples from healthy volunteers without evidence of an epithelial malignancy were examined. Peripheral blood was drawn from the middle of vein puncture after the first 3 ml of blood were discarded. This precaution was undertaken in order to avoid contamination of the sample with epithelial cells from the skin during sample collection and to assure a high specificity of the method. We next tested technical feasibility determining protein expression by immunofluorescence (IF) and DNA amplification by FISH in isolated CTCs. We evaluated the range of expression seen in CTCs by presence or absence of staining using an anti-CK and EGFR antibody. Moreover, CD133 (AC133, Isotype: mouse IgG) (Miltenyi Biotec) expression in CTCs was analyzed by IF. TOP2A and HER2 amplification by FISH were determined in isolated CTCs. In the same way, CTCs negatives to CK expression were evaluated to EGFR, TOP2A/HER2 and CD133 expression (FIG. 1).

HUVEC cell line was used as positive control for VIM and Slug expression. We evaluated their expression by presence or absence of staining using anti-VIM and anti-Slug antibodies (Santa Cruz Biotechnology) (FIG. 2A). SKBR3 tumor cell line was used as negative control for both markers (FIG. 3A).

1.4. EGFR Expression in CTCs

EGFR-positive cells were identified by immunocytochemistry and the signal was detected by chromogenic and fluorescent detection, respectively. Presence of CK cells were revealed by incubation with freshly prepared Fast Red TR/Naphthol AS-MX substrate solution and identified under a direct light microscope. Slides were washed once with PBS and stained with Mayer's haematoxylin solution (Sigma). EGFR⁺ cells were revealed by incubation with primary monoclonal anti-human EGFR (Dako) (dilution 1:25), followed by incubation with Alexa Flour 350 (Molecular Probes. Invitrogen). Epithelial tumor cells were identified and enumerated based on their red staining for CK-positive cells and blue staining for EGFR⁺ cells. SKBR3 tumor cell line was used as positive control for EGFR (FIG. 3A). Identification and counting were done with a computerized fluorescence microscope Zeiss AXIO Imager.

1.5. Vimentin and Slug Expression in CTCs

We selected the subpopulation of patients CTCs negatives to CK expression, after the selection by the immunomagnetic multi-CK enrichment method. These slides negatives for CK expression were, then, stained with conjugated VIM-FITC (mouse monoclonal antibody and Slug (rabbit polyclonal antibody) (FIG. 1). Slug expression was detected using anti-rabbit Alexa Fluor 633 a (Molecular Probes) for double immunofluorescence experiments, following the laboratory requirements (FIG. 1). Briefly, the slides were immersed in PBS for 5 minutes to hydrate the cells. Then, they were washed for 10 minutes in a solution of 0.2% NP40 in 2×SSC buffer at 70° C. Vimentin expression was revealed by incubation with an anti-VIM-FITC conjugated antibody for 45 minutes. After first staining step, the samples were washed twice and samples incubate with rabbit anti-Slug antibody for 45 minutes, followed by incubation with Alexa Fluor 633 anti-rabbit for 45 minutes. Finally, CD45 expression was revealed by incubation with an Alexa 405, to detect hematopoietic cells. Cells were considered EMT-positive if VIM⁺ and/or Slug+, CD45⁻. After the identification of these markers, nuclear staining with DAPI was performed. Specific staining for VIM and Slug was easily distinguished because of the differential intracellular distribution of the examined molecules and the combination of the double IF. Identification and counting were done with a computerized fluorescence microscope Zeiss AXIO Imager.

1.6. EMT Induction by TGFβ1 and/or EGF

2×10⁵ MCF-7 cells were seeded a day prior to starting the treatment at ˜30-40% confluence in 6 well plates, then stimulated with recombinant human TGFβ₁ (Santa Cruz Biotechnology) and/or EGF (Abcam, Cambridge) in DMEM without FBS medium at 0, 5 and 10 ng/ml or 0 and 20 ng/ml, respectively.

1.7. Western Blotting Analysis

Protein extraction and Western blot analysis were performed in MCF-7 cells induced or non-induced with TGFβ1 and EGF. MCF-7 cells (6×10⁶) were plated onto 75 cm² flasks and cultured overnight, followed by incubation with TGFβ1 and/or EGF at several concentrations (0, 5 and 10 ng/ml for TGFβ1 and 0 or 20 ng/ml for EGF). After 72 h of treatment cells were lysed in sample buffer (62.76 mM Tris-HCl pH 6.8, 5% 2-mercaptoethanol, 2% SDS, 10% glycerol, 0.5% bromophenol blue, and 100 mM dithiothreitol). Proteins (30 μg) were separated by SDS-PAGE (10%) in a Mini Protean II cell (Bio-Rad, Hercules, Calif.) and were transferred to a nitrocellulose membrane (80 V at room temperature for 30 min). Blots were treated with blocking solution (PBS TWIN 0.5% non-fat milk) for 1 h at room temperature and then reacted with primary antibody against VIM, Slug, Bcl-2, caspase 9, multi-CK, TGFβ R2 (Abcam, Cambridge) and EGFR (Santa Cruz Biotechnology, CA) at 1:1000 dilution overnight at 4° C. Then, membranes were washed and reincubated for 1 h with a horseradish peroxidase-conjugated anti-IgG (Abcam, Cambridge) diluted at 1:1000. Protein-antibody complexes were visualized by enhanced chemiluminescence (ECL, Bonus, Amersham, and Little Chalfont). Relative amounts of β-actin (Santa Cruz Biotechnology, CA) in treated and non-treated samples were determined by densitometric analysis using Quantity One 1-D analysis software (Bio-Rad).

1.8. Caspase-3, -7 and -8 Activities

Caspase-3, -7 and -8 activities were measured using Caspase-Glo® 3/7 and Caspase-Glo® 8 Assay kits (Promega, Madison, Wis., USA) according to manufacturer's instructions. Briefly, MCF-7 cells were seeded at 5×10⁴ cells/well in 96-well, white-walled plates and treated with TGFβ1 and/or EGF after cells were grown to 50% confluence. After incubation for 72 hours an equal volume of Caspase-Glo reagent was added to the culture medium. The plates were shaken at 500 rpm for 30 sec, incubated for 1 h, and the luminescence that is proportional to caspase 3/7 and 8 activities was determined by luminometer. Data are presented as the mean±SD from three replicates.

1.9. Would Healing Assay

Migration of MCF-7 cells treated with TGFβ₁ and/or EGF for 72 h was measured using the in vitro wound-healing assay. Cells were seeded into 6-well plates and grown to 80% confluence. Wounds were created by scraping monolayer cells with a 200 μl pipette tip and non-adherent cells washed off with medium. At 0, 24, 48 and 72 h after the creation of wounds, cells were observed with a 10× objective in an Axiovert 40 CFC Zeiss (Carl Zeiss meditec group, Germany) photomicroscope. Images were acquired with an AxioCam ICc3 Zeiss (Carl Zeiss meditec group, Germany) color digital camera. Wound distances were measured at each time point and expressed as the average percent of wound closure by comparing the zero time. Image-Pro Plus 6.0 software was used to quantify the wound area. All experiments were plated in triplicate wells and were carried out at least three times.

1.10. Confocal Microscopy

Confocal images were obtained using a Zeiss LSM 710 confocal/multi photon laser scanning microscope equipped with Argon/2 laser (458, 477, 488, 514 nm) and a Titanium Sapphire laser (750 nm). The cells were viewed with a 63× (NA 1.2) apochromatic water objective and images of different fields were taken. The microscope was set up to take multichannel images and the excitation and emission filter sets configured individually so that there is no fluorescence bleed-through between the channels. The argon (488 nm) laser with appropriate, emission filters was used for the visualization of Alexa Fluor 488. Alexa Fluor 488 was utilized to visualize EGFR, VIM and CK and an Alexa Fluor 633 was used to Slug. Adherent monolayer cultures of MCF7 cells were grown on glass coverslips for 72 hours with TGFβ1 (10 ng/ml) and with TGFβ1/EGF (10 ng/ml and 20 ng/ml, respectively) combination on culture slide (Becton Dickinson). Then, cells were washed and fixed with 3.7% formaldehyde in Dulbecco's PBS followed by permeabilization in 0.5% NP40. The cells were washed, blocked with 5% BSA in PBS, and incubated with primary antibody for overnight at 4° C. and with Alexa Fluor 488 or Alexa Fluor 633-conjugated secondary antibody for 1 hour. The slides were then washed with PBS for 15 min air dried, and mounted with Vectashield® mounting medium with 4′,6-diamidino-2-phenylindole (DAPI) (Cat# H-1200, Vector Laboratories). The images were captured using a spinning objective confocal microscope at ×60 magnification.

1.11. Statistical Analysis

The main objective was to investigate the VIM and Slug expression in CTCs in negatives samples to CK expression of non metastatic BC patients and correlate with clinical outcomes and with EGFR expression. The presence of at least 1 CTC per 10 ml was considered a positive result, according to the reported analytic detection limit of our assay [34]. We evaluated the range of expression of VIM and Slug seen in CK-negative CTCs by presence or absence of staining. The statistical analysis was performed using SPSS 14.0 software. Data are expressed as means or numbers (%). Categorical variables were compared by Pearson's chi-squared test (χ²), and continuous variables were compared by Student's t-test. Two-tailed P<0.05 values were considered statistically significant.

### Example 2

### Correlation of VIM and Slug Expression in CTCs with Clinical and Pathological Characteristics

Patients included in this study were consistent with an unselected early and locally advanced breast cancer population. Clinical-pathological characteristics were stratified according to the baseline VIM and Slug expression in CK-negative CTCs status (Table 1).

TABLE 1
Clinical-pathological characteristics according to the baseline status

| | | N(%) VIM+ | | N (%) VIM− | | p (χ²) | N (%) SLUG+ | | N (%) SLUG− | | p (χ²) |
|---|---|---|---|---|---|---|---|---|---|---|---|
| Age | ≦50 | 8 | (33.33) | 16 | (66.67) | 0.270 | 8 | (30.77) | 1 | | 0.305 |
| | >50 | 10 | (23.26) | 33 | (76.74) | | 9 | (20) | 8 | (69.23) | |
| | | | | | | | | | 36 | (80) | |
| Histology | Ductal | 17 | (29.31) | 41 | (70.69) | 0.258 | 16 | (25.81) | 46 | (74.19) | 0.309 |
| | Others | 1 | (11.11) | 8 | (88.89) | | 1 | (11.11) | 8 | (88.89) | |
| Clinical | ≦2 cm | 10 | (31.25) | 22 | (68.75) | 0.048* | 10 | (31.25) | 22 | (68.75) | 0.047* |
| Tumor Size | >2-5 cm | 3 | (12) | 2 | (88) | | 3 | (10.34) | 26 | (89.66) | |
| | >5 cm | 5 | (50) | 5 | (50) | | 4 | (40) | 6 | (60) | |
| Clinical | N0 | 17 | (29.82) | 40 | (70.18) | 0.182 | 16 | (26.67) | 44 | (73.33) | 0.196 |
| Node Status | N+ | 1 | (10) | 9 | (90%) | | 1 | (9.09) | 10 | (90.91) | |
| Grade | I | 2 | (13.33) | 13 | (86.67) | 0.224 | 2 | (13.33) | 13 | (86.67) | 0.361 |
| | II | 10 | (37.04) | 17 | (62.96) | | 9 | (32.14) | 19 | (67.86) | |
| | III | 4 | (20) | 16 | (80) | | 4 | (17.39) | 19 | (82.61) | |
| Hormonal | RH− | 3 | (21.43) | 11 | (78.57) | 0.442 | 3 | (20) | 12 | (80) | 0.490 |
| Status | RH+ | 15 | (28.30) | 38 | (71.70) | | 14 | (25) | 42 | (75) | |
| HER2 | HER2− | 16 | (28.7) | 41 | (71.93) | 0.460 | 16 | (26.23) | 45 | (73.77) | 0.248 |
| Status | HER2+ | 2 | (20.0) | 8 | (80) | | 1 | (10) | 9 | (90) | |
| P53 | P53− | 10 | (27.03) | 27 | (72.97) | 0.569 | 9 | (23.68) | 29 | (76.32) | 0.483 |
| Status | P53+ | 3 | (30) | 7 | (70) | | 3 | (30) | 7 | (70) | |
| KI67% | (KI67−) < 14% | 7 | (28) | 18 | (72.0) | 0.544 | 7 | (28) | 18 | (72) | 0.377 |
| | (KI67+) ≧ 14% | 11 | (26.19) | 31 | (73.81) | | 10 | (21.14 | 35 | (78.26) | |

Only, clinical tumor size status was the primary tumor features that correlated with VIM+ and Slug+ CTCs (P=0.048 and P=0.047, respectively). No significant correlation was found between VIM+/Slug+ expression in CTCs and clinical characteristics of patients, including age (<50 vs ≧50 years old), histology, nodal status (NO vs N1-2), grade (gI vs gII vs gIII), hormonal status (RH+ vs RH−), p53 status (positive vs negative) or KI67 (<14 vs ≧14). There was no correlation between VIM or Slug/CK-negative CTC status and breast cancer subtypes (luminal vs triple negatives/HER2 amplified) (data not shown).

### Example 3

### Correlation of VIM and Slug Expression in CK-Negative CTCs with EGFR, CD133, TOPO2/HER2 Biomarkers

To test the expression of EMT-like CTCs, 78 blood samples negative for multi-CK-specific markers were processed using CD45 (a leukocyte marker), VIM and Slug (EMT markers) by IF (FIG. 1).

For VIM analysis, only 67 samples were considered due to a background staining. From these samples, 18 (26.9%) showed VIM expression and 49 (73.1%) were negative. For Slug expression, only 71 samples were considered due to a background staining. 17 (23.9%) of these patients were positives for Slug expression and 54 (76.1%) were negative. Interestingly, when Slug was positive in CK-negative CTCs, VIM marker was co-expressed in 94.4% (17/18) of CK-negative CTCs. However, the expression of VIM not always co-expressed with Slug (1/18; 5.56%). Thus, VIM negative CTCs were also negative to Slug (49; 100%) (Table 2).

TABLE 2
Correlation of Vim and Slug status in Ck-negative CTCs

| | SLUG | | |
|---|---|---|---|
| VIM | SLUG− (N) (%) | SLUG−+ (N) (%) | TOTAL |

| | | | | | |
|---|---|---|---|---|---|
| VIM− | 49 | (100) | 0 | (0) | 49 |
| | | | | | P = 0.00 |
| VIM+ | 1 | (5.56) | 17 | (94.44) | 18 |

Also, we found that Slug showed a different cellular distribution. In most of samples, Slug staining was detected in the cytoplasm (FIG. 2B); however, some samples showed a nuclear staining pattern as well (FIG. 2C).

Additionally, in order to examine the correlation of EFGR, TOPO2/HER2 and CD133 with VIM and Slug expression, we studied the presence of these biomarkers in patients with CK-negative CTCs before any treatment (Table 3).

TABLE 3
Correlation between EMT markers (Vim and Slug) and EGFR, CD133 and TOPO2/HER2
in Ck-negative CTCs

| | EMT MARKERS | VIM+ N (%) | VIM− N (%) | p | SLUG+ N (%) | SLUG− N (%) | p |
|---|---|---|---|---|---|---|---|
| EGFR | EGFR+ | 8 (44.5) | 10 (55.5) | 0.044 | 8 (42.1) | 11 (57.9) | 0.030 |
| | EGFR− | 9 (18.4) | 40 (81.6) | | 9 (17.3) | 43 (82.7) | |
| CD133 | CD133+ | 0 (0) | 0 (0) | — | 0 (0) | 0 (0) | — |
| | CD133− | 18 (26.9) | 49 (73.1) | | 17 (24.0) | 54 (76.0) | |
| TOPO2/HER2 | TOPO2/HER2+ | 3 (37.5) | 5 (62.5) | 0.366 | 3 (25) | 9 (75) | 0.592 |
| | TOPO2/HER2− | 15 (25.4) | 44 (74.6) | | 14 (23.7) | 45 (763) | |

Of 67 patients analyzed for VIM and EGFR expression, we found that in samples EGFR⁻, 9 (18.4%) were VIM⁺ and 40 (81.6%) were VIM⁻. In samples EGFR⁺, 10 (55.5%) were VIM⁻ and 8 (44.5%) were VIM⁺ (P=0.044). Also, EGFR expression was analyzed in 71 patient positives for Slug marker. Nine (17.3%) samples were EGFR⁻/Slug⁺, 43 (82.7%) were EGFR⁻/Slug⁻ and 11 (57.9%) were EGFR⁺/Slug⁻ and 8 (42.1%) were EGFR⁺/Slug⁺ (P=0.030). For CD133 expression, we did not found correlation with EMT markers in CK-negative CTCs. Moreover, TOPO2/HER2 amplification was detected only in 8 of 67 patients and only 3 samples VIM⁺ (37.5%; P=0.366). Slug expression was detected in 3 of 12 (25%) TOPO2/HER2⁺ patients (P=0.592) (Table 3).

### Example 4

### Modifications in CK, EGFR and Apoptotic Markers after Induction of EMT Phenomenon in MCF-7 Cells

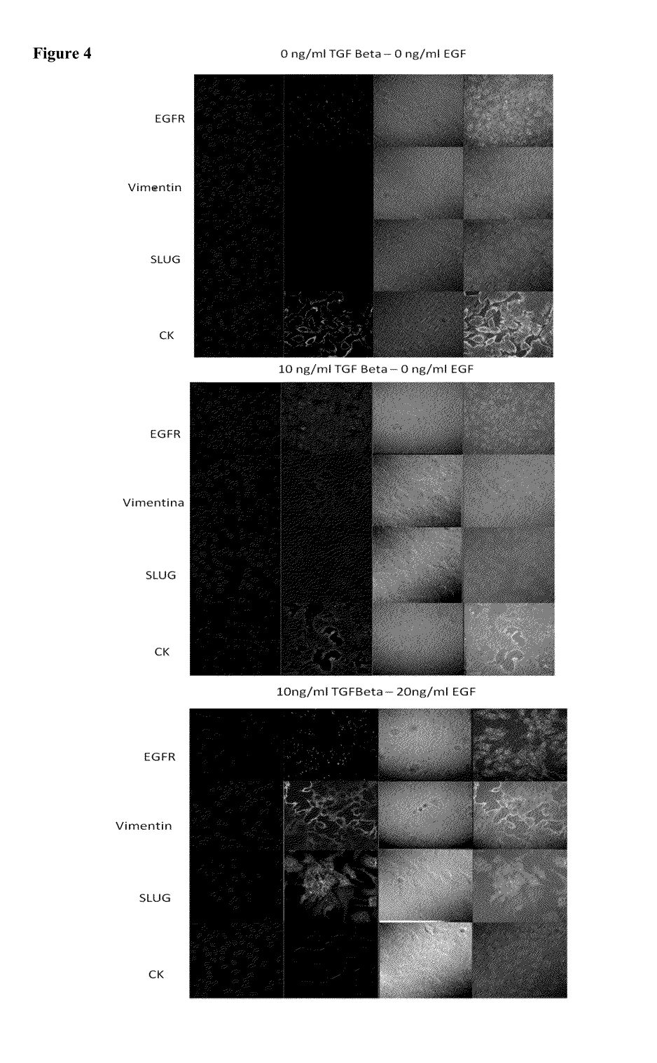

To further explain the relationship between EGFR expression in CK-negative CTCs and EMT process we developed an experimental model using MCF-7 tumor cells. MCF-7 cells were stimulated with TGFβ₁ and/or EGF at several concentrations for 72 h. Firstly, we analyzed by western blotting the switch in the pattern of expression of VIM, Slug, Pan-CK and EGFR status. Additionally, we analyzed changes in the apoptosis mediators Bcl-2 and pro-caspase 9 (FIG. 4). Our results showed that the induction with TGFβ₁/EGF combination maintained EGFR activation which was accompanied by a significant enhanced expression of both VIM and Slug markers and the complete inhibition in Pan-CK expression. Moreover, TGFβ₁/EGF combined treatment involved a significant induction in Bcl-2 expression and no modifications in pro-caspase-9 (FIG. 4A, 4B). Furthermore, caspase-3, -7 and -8 activities increased after induction with 10 nM TGFβ₁ and significantly decreased when EGF was added (FIG. 4C). The significant Bcl-2 and VIM and Slug induction together the caspase 3/7 and 8 decreasing demonstrate the inhibition of extrinsic apoptotic pathway after acquisition of EMT phenotype in EGFR⁺/CK-negative cells.

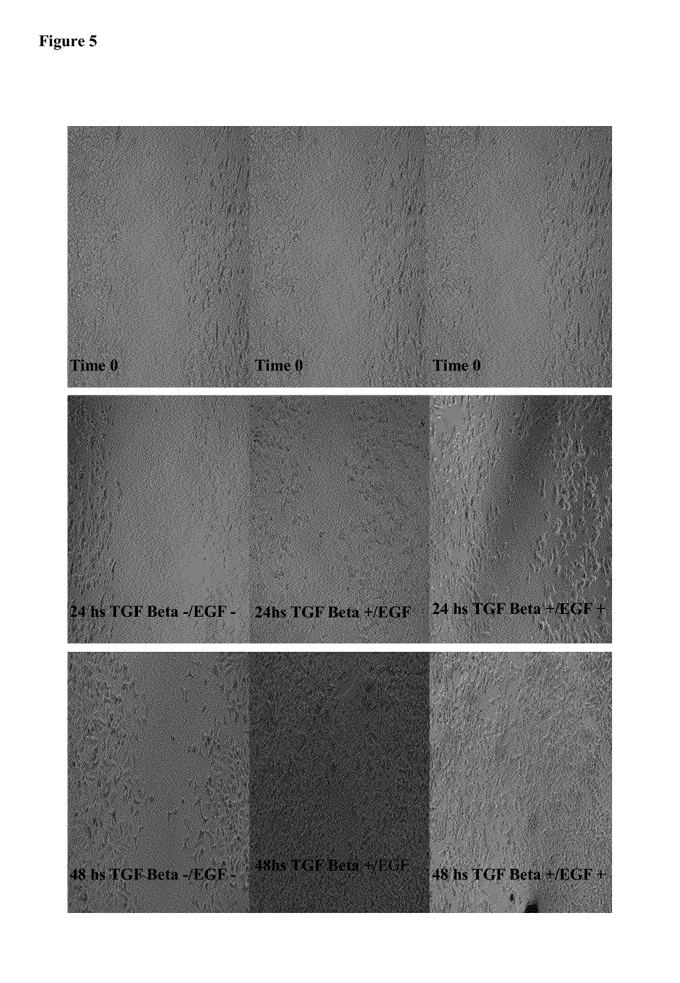

Finally, immunofluorescence analysis confirmed data obtained by western blot. The morphologic changes of MCF-7 cells after transformation with TGFβ₁ and EGF included loss of cell adhesion, reduced cell-cell contact, and increased pseudopodia (FIG. 5 A-C). Moreover, confocal microscopy studies showed an increased and robust expression of VIM and Slug, which was located in both nucleus and cytoplasm and the total disappearance of CK staining after TGFβ₁/EGF combined treatment. These results confirm the loss of epithelial phenotype and the acquisition of mesenchymal markers in CK negative/EGFR⁺ MCF7 cells (FIG. 5).

### Example 5

### Treatment with TGFβ₁ or TGFβ₁/EGF Promotes Cell Motility

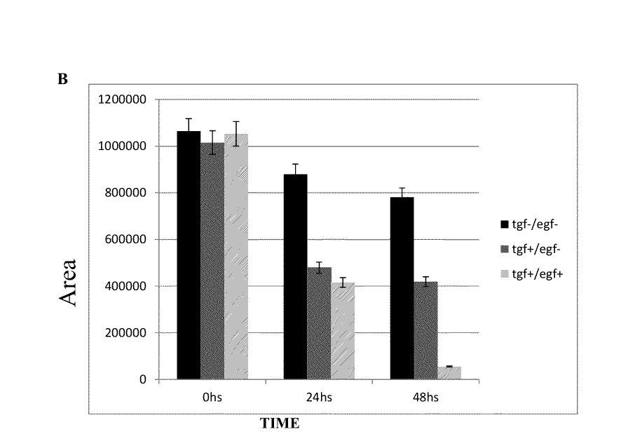

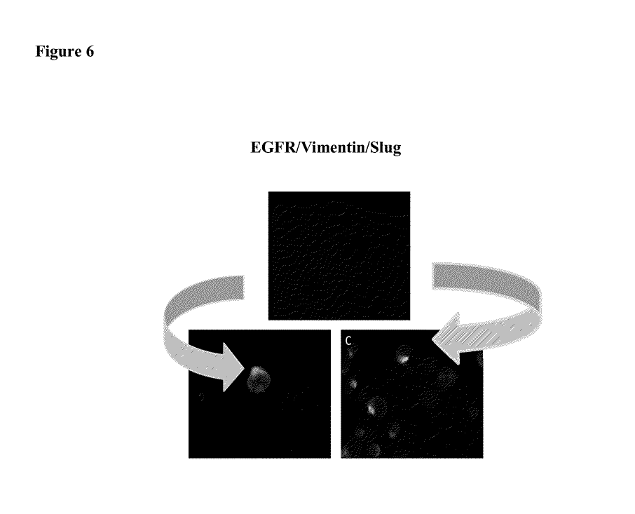

Since EMT is generally associated with a migratory phenotype that is indispensable for cancer metastasis, we performed wound healing assays in MCF-7 cells treated with TGFβ₁ or TGFβ₁/EGF. Our purpose was to demonstrate that continued activation of EGFR and the corresponding CK inhibition is associated with a higher migratory potential. Our results showed that TGFβ₁ or TGFβ₁/EGF exposition significantly increased the motility in MCF7 cells in a time-dependant manner with respect to control non-induced cells (FIG. 6 A, B). This enhanced migration was more evident in the combined treatment in comparison with the TGFβ₁ induction alone (FIG. 6B).

### Example 6

### EGFR and Relapse Correlation

After a systemic treatment of 78 patients analyzed, only 25 were CK-negatives CTCs and 53 changed from CK negative to CK positive CTCs. Of these CK negatives CTCs, 2 (66.7%) of the patients EGFR⁺ showed a relapse of the disease versus 1 (33.33%) without EGFR expression (P=0.032) (Table 4).

TABLE 4
Relationship between EGFR expression and relapse in breast
cancer patients negative to CK expression before treatment

| | | | |
|---|---|---|---|
| CTC-EGFR | No relapse | Relapse | P |

| | | | | | |
|---|---|---|---|---|---|
| CTC−/EGFR− | 21 | (95.45%) | 1 | (4.55%) | 0.032 |
| CTC−/EGFR+ | 1 | (33.33%) | 2 | (66.66%) | |

### Example 7

### Correlation of VIM and Slug Expression in CK-Negative CTCs with EGFR, CD133, Biomarkers

7.1. Colon Cancer

From March 2013 to June 2014, 78 metastatic colon cancer (mCRC) patients with Stage IV were identified from San Cecilio Hospital—Oncology Medical Unit. Medical charts of these patients were reviewed and clinical details were included in a database. The inclusion criteria were histological diagnostic of mCRC with availability to collect tissue for biomarker studies. All patients were treated with FOLFOX plus Bevacizumab during their mCRC first line of treatment. Medical charts of these patients were reviewed and clinical details of these patients were included in a database. This translational study was approved by the Ethics Reviewer Committees of the San Cecilio Hospital. Informed consent was obtained from all patients and healthy volunteers. The local ethics committee approved this study and eligible patients signed an approved informed consent. Medical charts of these patients were reviewed and clinical details were included in a database

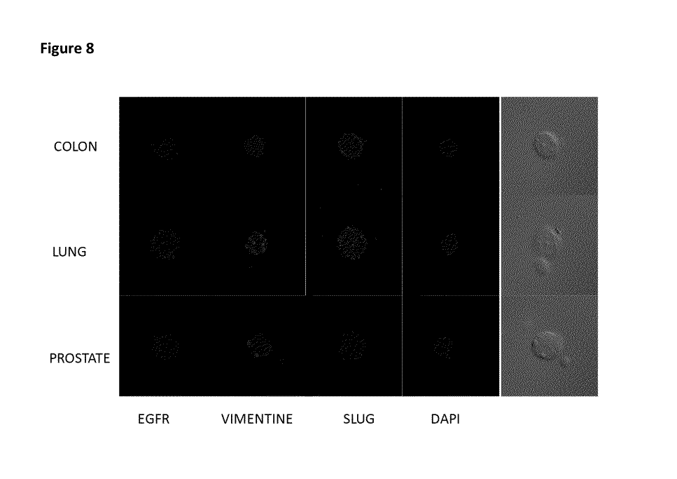

To test the expression of EMT-like CTCs, 78 blood samples negative for multi-CK-specific markers were processed using CD45 (a leukocyte marker), VIM and Slug (EMT markers) by IF (FIG. 8).

From these samples, 25 (32.1%) showed VIM expression and 53 (67.9%) were negative. For Slug expression, 22 (28.2%) of these patients were positives for Slug expression and 56 (71.8%) were negative (see table 5 below).

TABLE 5
MESTASTATIC COLON CANCER

| | EMT MARKERS | VIM+ N (%) | VIM− N (%) | p | SLUG+ N (%) | SLUG− N (%) | p |
|---|---|---|---|---|---|---|---|
| EGFR | EGFR+ | 12 (44.5) | 15 (55.5) | 0.05 | 10 (38.4) | 16 (61.6) | 0.030 |
| | EGFR− | 13 (25.4) | 38 (74.6) | | 11 (24) | 35 (76) | |
| CD133 | CD133+ | 1 (33.3) | 2 (66.7) | — | 0 (0) | 0 (0) | — |
| | CD133− | 17 (26.6) | 47 (73.4) | | 17 (24.0) | 54 (76.0) | |

Of 78 patients analyzed for VIM and EGFR expression, we found that in samples EGFR⁻, 13 (25.4%) were VIM⁺ and 38 (74.6%) were VIM⁻. In samples EGFR+, 15 (55.5%) were VIM⁻ and 12 (44.5%) were VIM⁺ (P=0.05). Also, EGFR expression was analyzed in 72 patient for Slug marker. Eleven (24%) samples were EGFR⁻/Slug⁻, 35 (76%) were EGFR⁻/Slug⁻ (P=0.030). For CD133 expression, we did not found correlation with EMT markers in CK-negative CTCs. (see table 5).

7.2. Lung Cancer

73 stage I to IV NSCLC patients (40 stage I to III and 33 stage IV) were enrolled from the Surgery Unit of the University Hospital of Granada from March 2012 to September 2013. The inclusion criteria were the histological diagnosis of NSCLC and the availability of tissue for biomarker studies. The local ethics committee approved this study and eligible patients were selected after the acquisition of informed written consent. Surgical procedures and systemic therapy were given at the discretion of the treating physician with or without targeted therapy NSCLC patients. The medical charts of these patients were reviewed and their clinical details were included in a database.

Tumor specimens from archival tumor biopsies of each patient were obtained and analyzed for different markers.

7.2.1. Operable Lung Cancer

To test the expression of EMT-like CTCs, 40 blood samples negative for multi-CK-specific markers were processed using CD45 (a leukocyte marker), VIM and Slug (EMT markers) by IF (FIG. 8). From these samples, 13 (32.5%) showed VIM expression and 27 (67.5%) were negative. For Slug expression, 12 (30%) of these patients were positives for Slug expression and 28 (70%) were negative (see table 6).

TABLE 6
OPERABLE LUNG CANCER

| | EMT MARKERS | VIM+ N (%) | VIM− N (%) | p | SLUG+ N (%) | SLUG− N (%) | p |
|---|---|---|---|---|---|---|---|
| EGFR | EGFR+ | 5 (41.7) | 7 (58.3) | 0.044 | 5 (42.1) | 7 (57.9) | 0.045 |
| | EGFR− | 8 (28.6) | 20 (71.4) | | 7 (17.3) | 21 (82.7) | |
| CD133 | CD133+ | 1 (50) | 15 (0) | — | 0 (0) | 0 (0) | — |
| | CD133− | 16 (42.1) | 22 (57.9) | | 13 (32.5) | 27 (67.5) | |

Of 40 patients analyzed for VIM and EGFR expression, we found that in samples EGFR⁻, 8 (28.6%) were VIM⁺ and 20 (71.4%) were VIM⁻. In samples EGFR⁺, 7 (58.3%) were VIM⁻ and 5 (41.7%) were VIM⁺ (P=0.044). Also, EGFR expression was analyzed in 40 patients for Slug marker. Seven (17.3%) samples were EGFR⁻/Slug⁺, 21 (82.7%) were EGFR⁻/Slug⁻ (P=0.045). For CD133 expression, we did not found correlation with EMT markers in CK-negative CTCs (see table 6).

7.2.2. Metastatic Lung Cancer

To test the expression of EMT-like CTCs, 33 blood samples negative for multi-CK-specific markers were processed using CD45 (a leukocyte marker), VIM and Slug (EMT markers) by IF (FIG. 8). From these samples, 11 (33.3%) showed VIM expression and 22 (66.7%) were negative. For Slug expression, 10 (30.3%) of these patients were positives for Slug expression and 23 (69.7%) were negative (see table 7).

TABLE 7
METASTATIC LUNG CANCER

| | EMT | | | | | | |
|---|---|---|---|---|---|---|---|
| | MARKERS | VIM+ N (%) | VIM− N (%) | p | SLUG+ N (%) | SLUG− N (%) | p |
| EGFR | EGFR+ | 4 (40) | 6 (60) | 0.044 | 4 (88.9) | 1 (11.1) | 0.032 |
| | EGFR− | 7 (30.4) | 16 (70.6) | | 6 (28.6) | 15 (71.4) | |
| CD133 | CD133+ | 5 (100) | 0 (0) | 0.039 | 5 (100) | 0 (0) | 0.039 |
| | CD133− | 1 (3.6) | 27 (96.4) | | 1 (3.6) | 27 (96.4) | |

Of 33 patients analyzed for VIM and EGFR expression, we found that in samples EGFR⁻, 7 (30.4%) were VIM⁺ and 16 (70.6%) were VIM⁻. In samples EGFR⁺, 6 (60%) were VIM⁻ and 4 (40%) were VIM⁺ (P=0.044). Also, EGFR expression was analyzed in 33 patient for Slug marker. Six (28.6%) samples were EGFR⁻/Slug⁺, 15 (71.4%) were EGFR⁻/Slug⁻ (P=0.032). For CD133 expression, we found that in samples CD133⁻, 1 (3.6%) were VIM⁺ and 27 (96.4%) were VIM⁻. In samples CD133⁺, 0 (0%) were VIM⁻ and 5 (100%) were VIM⁺ (P=0.039). Also, CD133 expression was analyzed positives for Slug marker. One (3.6%) samples were CD133⁻/Slug⁺, 27 (96.4%) were cd133⁻/Slug⁻ (P=0.039).

7.3. Prostate Cancer

59 stage I to I-III prostate cancer (PC) patients were enrolled from the Uroglogy Unit of Virgen de las Nieves Hospital of Granada from March 2013 to September 2014. The inclusion criteria were the histological diagnosis of prostate cancer and the availability of tissue for biomarker studies. The local ethics committee approved this study and eligible patients were selected after the acquisition of informed written consent. Surgical procedures and treatment were given at the discretion of the treating physician with or without targeted therapy PC patients. The medical charts of these patients were reviewed and their clinical details were included in a database.

Tumor specimens from archival tumor biopsies of each patient were obtained and analyzed for different biomarkers.

To test the expression of EMT-like CTCs, 59 blood samples negative for multi-CK-specific markers were processed using CD45 (a leukocyte marker), VIM and Slug (EMT markers) by IF (FIG. 8). From these samples, 13 (22%) showed VIM expression and 46 (78%) were negative. For Slug expression, 7 (11.5%) of these patients were positives for Slug expression and 54 (88.5%) were negative (see table 8)

TABLE 8
PROSTATE CANCER

| | EMT | | | | | | |
|---|---|---|---|---|---|---|---|
| | MARKERS | VIM+ N (%) | VIM− N (%) | p | SLUG+ N (%) | SLUG− N (%) | p |
| EGFR | EGFR+ | 4 (40) | 6 (60) | 0.044 | 3 (21.4) | 11 (78.6) | 0.030 |
| | EGFR− | 9 (18.4) | 40 (81.6) | | 4 (8.5) | 43 (91.5) | |
| CD133 | CD133+ | 10 (76.9) | 3 (23.1) | — | 7 (46.6) | 8 53.4) | — |
| | CD133− | 20 (43.5) | 26 (56.5) | | 20 (44.4) | 25 (55.6) | |

Of 59 patients analyzed for VIM and EGFR expression, we found that in samples EGFR⁻, 9 (18.4%) were VIM⁺ and 40 (81.6%) were VIM⁻. In samples EGFR⁺, 6 (60%) were VIM⁻ and 4 (40%) were VIM⁺ (P=0.044). Also, EGFR expression was analyzed in 61 patients for Slug marker. In samples EGFR⁺, 3 (21.4%) were SLUG+ and 11 (78.6%) were SLUG⁻ (P=0.030). Four (8.5%) samples were EGFR⁻/Slug⁺, 43 (91.5%) were EGFR⁻/Slug⁻ (P=0.03). For CD133 expression, we did not found correlation with EMT markers in CK-negative CTCs (see table 8)

1. A method for prognosticating cancer in a human subject with cancer comprising the steps of:
a. obtaining one or more biological samples from the subject suffering from cancer comprising circulating tumour cells;
b. measuring the overall expression pattern or level of the following biomarkers in the circulating tumour cells obtained from the one or more biological samples of the subject: (i) CK (cytokeratin) and (ii) (A) EGFR (epidermal growth factor receptor) or (B) EMT antigens VIM and Slug or (C) both EGFR and EMT antigens VIM and Slug; and
c. comparing the overall expression pattern of the above mentioned biomarkers in the circulating tumour cells from the biological sample of the subject suffering from cancer with the overall expression pattern of the biomarkers from the circulating tumour cells in a control biological sample, wherein (i) overexpression or increase expression of EGFR in CK negative (underexpressed) circulating tumour cells, (ii) overexpression of EMT antigens VIM and Slug or increase expression in CK negative (underexpressed) circulating tumour cells, or (iii) overexpression or increase expression of EMT antigens VIM and Slug and of EGFR in CK negative (underexpressed) circulating tumour cells is indicative of Progression Free Survival (PFS) and/or Overall Survival (OS).

2. (canceled)

3. (canceled)

4. The method of claim 1, wherein the one or more biological samples are selected from the group consisting of plasma sample, a serum sample, a blood sample, a tissue sample, and a faecal sample.

5. The method of claim 1, wherein the expression level of the biomarkers is measured by microarray expression profiling, PCR, reverse transcriptase PCR, reverse transcriptase real-time PCR, quantitative real-time PCR, end-point PCR, multiplex end-point PCR, cold PCR, ice-cold PCR, mass spectrometry, in situ hybridization (ISH), multiplex in situ hybridization, or nucleic acid sequencing.

6. The method of claim 1, wherein the cancer is a solid tumour of epithelial origin.

7. The method of claim 1, wherein the cancer is selected from the list consisting of colon cancer, lung cancer, breast cancer and prostate cancer.

8. The method of claim 1, wherein the method is used for treating a patient suffering from cancer, selecting an anti-neoplastic agent therapy for a patient suffering from cancer, stratifying a patient to a subgroup of cancer or for a cancer therapy clinical trial, determining resistance or responsiveness to a cancer therapeutic regimen, developing a kit for diagnosis of cancer, or any combinations thereof.

9. The method of claim 1, further comprising the step of using the overall expression pattern or level of the biomarkers for prognosis, treatment guidance, or monitoring response to treatment of the cancer.

10. A kit for a prognosis of cancer or for predicting the response of a human subject suffering from cancer to therapy with an EGFR inhibitor comprising: biomarker detecting reagents for determining a differential expression level of EMT antigens VIM and Slug, EGFR and CK in circulating tumour cells obtained from biological samples.

11. The kit of claim 10, further comprising instructions for use in diagnosing risk for cancer, wherein the instructions comprise step-by-step directions to compare the expression level of the biomarkers, when measuring the expression of a sample obtained from a subject suspected of having neoplasia with the expression level of a control sample.

12. The kit of claim 10, further comprising tools, vessels and reagents necessary to obtain samples from a subject selected from the group consisting of one or more biological fluids, a plasma sample, a serum sample, a blood sample, a tissue sample, or a fecal sample.

13. A method of predicting the response of a human subject to therapy with an EGFR inhibitor, wherein the subject is suffering from cancer, comprising the steps of:
a. obtaining one or more biological samples from the subject suffering from cancer comprising circulating tumour cells;
b. measuring the overall expression pattern or level of the following biomarkers in the circulating tumour cells obtained from the one or more biological samples of the subject: (i) CK (cytokeratin) and (ii) (A) EGFR (epidermal growth factor receptor) or (B) EMT antigens VIM and Slug or (C) both EGFR and EMT antigens VIM and Slug; and
c. comparing the overall expression pattern of the above mentioned biomarkers in the circulating tumour cells from the biological sample of the subject suffering from cancer with the overall expression pattern of the biomarkers from the circulating tumour cells in a control biological sample, (i) overexpression or increase expression of EGFR in CK negative (underexpressed) circulating tumour cells, (ii) overexpression of EMT antigens VIM and Slug or increase expression in CK negative (underexpressed) circulating tumour cells, or (iii) overexpression or increase expression of EMT antigens VIM and Slug and of EGFR in CK negative (underexpressed) circulating tumour cells is indicative of no response and/or partial response of the subject.

14. (canceled)

15. (canceled)

16. The method of claim 13, wherein the EGFR inhibitor is selected from the group consisting of Gefitinib, Erlotinib, Cetuximab, lapatinib, pannitumumab and trastuzumab.

17. A method for allocating a human subject suffering from cancer in one of two groups, wherein group 1 comprises subjects identifiable by the method according to claim 13 as predicted to show any one or more of:
(i) No response; or
(ii) partial response;
and wherein group 2 represents the remaining subjects.

18. A pharmaceutical composition comprising an anti-EGFR agent, for treating a human subject of group 2 as identifiable by the method of claim 17.

19. The pharmaceutical composition of claim 18 wherein the anti-EGFR agent is selected from the group consisting of Gefitinib, Erlotinib, Cetuximab, lapatinib, pannitumumab and trastuzumab.

20. The method of claim 1, wherein the cancer is a non-metastatic solid tumour of epithelial origin.
